(19) United States
(12) Patent Application Publication
(10) Pub. No.: US 2003/0194693 A1
(43) Pub. Date: Oct. 16, 2003

(54) Method of prevention of lysosomal leakage in eukaryotic cells by using 6-methyluracil based water-soluble compounds and method of producing thereof

(76) Inventor: Alexander Y. Oksman, Brooklyn, NY (US)

Correspondence Address: Boris Leschinsky, P.O. Box 72, Waldwick, NJ, 07463, US

(21) Appl. No.: 10/371,807

(22) Filed: Feb. 21, 2003

Related U.S. Application Data
(60) Provisional application No. 60/358,962, filed on Feb. 25, 2002.

(51) Int. Cl. A01N 1/00; A01N 1/02; C12N 1/18; A61K 31/7072

(52) U.S. Cl. 435/2; 514/49; 435/254.2; 435/1.1

(57) ABSTRACT

A composition and method of preventing a lysosomal leakage from eukaryotic cells is based on a 6-methyluracil based water-soluble compound. The cells are being exposed to a water solution of such compound for the duration of a sub-optimal condition such as a temperature shock, starvation, radiation, etc. The optimum concentration of the compound is ranging from 1 to 1000 micrograms per milliliter of a solution. The preferred concentration is from 10 to 500 mkg/ml. The most advantageous composition from the group is a complex of 2,4-dihydroxy-6-methylpyrimidine with N-methyl-D-glucamine. Besides cell preservation, the composition is useful for a treatment of a variety of medical conditions characterized by excessive or inappropriate apoptosis such as ischemia, type 1 diabetes, stroke, Alzheimer's, and Parkinson's diseases. The composition is also useful in yeast production, food preservation, and other applications.

### CROSS-REFERENCE DATA

This application claims the priority from a corresponding Provisional Application No. 60/358,962 filed 02/25/2002 and a Disclosure Document No. 499774 filed Sep. 11, 2001, both of which are incorporated herein in its entirety by reference.

### BACKGROUND

Eukaryotic cells include a large range of cells from the simplest yeast cells to very complex mammalian cells. All such cells have a nucleus that includes genomic DNA.

Apoptotic cell death under various sub-optimal conditions can be caused by a variety of factors. Examples of sub-optimal conditions include freezing, thawing, lyophilization, contact with various chemical compounds (such as toxins), starvation, growth in a saturated state, death of host organism, etc. Lysosomal leakage is believed to be one of the early steps leading to eukaryotic cell death [see for example Guo-Jiang Zhang et al. The direct cause of photomage-induced lysosomal destabilization, Bioch Biohys Acta, 1997, 1326, p.75-82; and G Majno et al. Apoptosis, oncosis, and necrosis. An overview of cell death. Am J Pathol, 1995, 146;1, p.3-15]. Membrane destruction and the leakage from lysosomes of enzymes, proteases, proteolytic activity, etc. into cytoplasm causes irreparable cell damage and eventually leads to its apoptotic death (also referred in literature as apoptosis, programmed cell death, and physiological cell death).

Prevention or slowing the progress of apoptotic cell death is highly desirable and would have significant applications for clinical, medical, cosmetic and commercial purposes. It would improve the survival rate of cells or cell cultures, especially for those cultures exposed to less-than-optimal growing conditions or for cultures that are difficult to replicate. It would slow the rate of cell decay in some food products to extend the storage time, or lower the refrigeration temperature, allowable prior to their consumption. It would reduce damage to the tissue of organ transplants while being transported from donor to recipient patient. It is believed that a method of preventing lysosomal leakage would indeed break the apoptotic death cycle and preserve the cells. The need therefore exists for a method of preserving the lysosome membrane and preventing its leakage.

General cytoprotection and prevention of proteolytic tissue decay are just two of the most direct applications for such a method although other applications may be considered as well. It would, for example, be beneficial for the regeneration of skeletal muscles (trauma), heart muscles (myocardial, infarction), liver and kidney cells as well as for the treatment of different degenerative diseases (Parkinson's, Alzheimer's, etc.). It would improve the survival rate of cells undergoing nucleic transfer during cloning activities. It would improve the growth of primer cell cultures, such as skin cell grafts, neurons, etc. It would improve the survival rate of cells while in storage (sperm cells, embryos, stem cells, cells undergoing cryogenic preservation, lyophilization, etc.). It would also provide protection against the damage caused by radiation (such as X-rays).

### SUMMARY OF THE INVENTION

Accordingly, the object of the present invention is to provide a composition and methods of use allowing preventing a lysosomal leakage from a eukaryotic cell.

Another object of the invention is to provide a method for manufacturing of such a compound.

In accordance with the present invention, it has been discovered that 6-methyluracil based water-soluble compounds have a cytoprotection quality and can reduce or entirely prevent apoptosis of eukaryotic cells in sub-optimal conditions.

For the purposes of this description, the term “sub-optimal condition” means any single or combination of factors causing cell death such as extreme hot or cold temperature, freezing, radiation, mechanical or chemical stress, starvation, exposure to toxic environment, lyophilization, trauma, etc.

According to the invention, exposure of eukaryotic cells to a water-based solution of 6-methyluracil in a particular concentration range prior and/or for at least a portion of the duration of a sub-optimal condition can prevent lysosomal leakage and therefore prevent apoptosis of such cells. It is advantageous to expose the cells for the entire duration of the sub-optimal condition. It has been discovered that the best concentration is generally from about 1 to about 1000 mkg/ml (micrograms per milliliter) of a 6-methyluracil compound in a biocompatible water solution. In a preferred form, this concentration is from about 10 to about 500 mkg/ml. Once the sub-optimal condition is no longer present, the cells can be withdrawn from the solution of the invention and the protective action would cease.

One particular advantageous representative of the 6-methyluracil family is a complex of 2,4-dihydroxy-6-methylpyrimidine with N-methyl-D-glucamine.

### BRIEF DESCRIPTION OF THE DRAWINGS

FIG. 1 is a chart demonstrating survival of rat brain cells in the presence and absence of Glucural over 7-day period.

FIG. 2 is a chart indicating the presence of neurons in the same experiment with and without Glucural present.

### DETAILED DESCRIPTION OF THE INVENTION

It has been unexpectedly discovered that water-soluble compounds based on 6-methyluracil could be efficiently used to prevent lysosomal leakage.

6-methyluracil based compounds are generally known as non-steroid anti-inflammatory drugs. The water-soluble form of it was synthesized in the 1970's and was used exclusively as an anti-inflammatory agent. Water-soluble compounds based on 6-methyluracil have important practical advantages. High level of water-solubility is beneficial when a substance is being considered for intravenous injection or for its ability to pass through the cell membrane.

Compounds of that nature can be dissolved in water or water-based solutions. For the purposes of this description, the terms “water”, “water-based solution”, “physiological solution” are used interchangeably to generally mean a biocompatible water solution creating a friendly environment for long-term survival of eukaryotic cells.

There are many similar compounds that can be generated under this general category. Detailed evaluation however was conducted on one particular representative compound, namely a complex of 2,4-dihydroxy-6-methylpyrimidine with N-methyl-D-glucamine, of the formula:

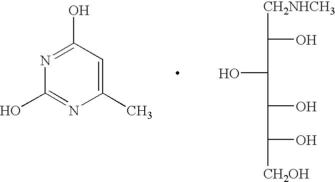

which is called Glucural (also sometimes referred to as Amygluracil and MMD).

Glucural's ability to significantly reduce lysosomal leakage, thereby preventing or slowing the progress of apoptotic cell death, has many applications, such as a cyto-protector and as an agent to reduce proteolytic decay. It would improve the survival rate of cells or cell cultures, especially those cultures exposed to less-than-optimal growing conditions or for cultures that are difficult to replicate, such as primer cell cultures. It would improve the survival rate of lyophilized yeast cultures. It would improve the survival rate of cells undergoing nucleic transfer during cloning activities. It would improve the survival rate of cells while in storage (sperm cells, embryos, stem cells, cells undergoing cryogenic preservation, etc.). It would also provide protection against the damage caused by radiation (such as X-rays). It would, for example, be beneficial for the regeneration of skeletal muscles (trauma), heart muscles (myocardial infarction), liver and kidney cells, etc. as well as for the treatment of different degenerative diseases (Parkinson's, Alzheimer's, etc.) and in cosmetic applications for preservation of skin. When used as a food preservative, it would slow the rate of cell decay to extend the storage time (or lower the refrigeration temperature) allowable prior to their consumption. It would reduce damage to the tissue of organ transplants while being transported from donor to recipient patient.

In addition to its ability to prevent lysosomal leakage, it has several qualities that make it desirable for use as a cyto-protector or to reduce proteolytic decay. It is highly water-soluble (more than 30 mg/ml compared to less than 3 mg/ml for thalidomides). This high level of water-solubility is beneficial when the substance is being considered for intravenous injection or for its ability to pass through the cell membrane. Glucural is also low in toxicity, (its LD₅₀ is about 2 g/kg for mice, or the equivalent of 100 g for a 50-kg person). Glucural is a small compound that is relatively easy to digest when taken orally, so applications as a food additive should be possible without concern about harmful doses building up in the body of a consumer. Glucural is an inexpensive compound to manufacture and its shelf life is more than 10 years at ambient temperatures.

It is believed that Glucural has the potential to be the most effective lysosomal membrane stabilizer known today. At the present time, different enzyme inhibitors are used to slow or suppress apoptosis. These known enzyme inhibitors utilize a different mechanism of action than Glucural, and can be used in conjunction with Glucural to improve results.

Clinical and Biological Uses of Glucural:

Glucural can be used to improve the viability of eukaryotic cell cultures when stored or grown. It is beneficial in improving the viability of stored sperm cells, embryos (humans, cattle, etc.) and cells of green plants (the viability of green plant cells after storage in a frozen state). It is very helpful to improve cell viability during the storage of yeast cells (including after lyophilization) and during all fermentation processes. Glucural can be very useful with many primer cell cultures, including for difficult to grow cultures, such as liver cells, neurons, etc. It can also be used to improve the success rate of nucleic transfer in cloning procedures.

In-vitro Evaluations of Glucural for Protection of Various Eukaryotic Cells in Sub-Optimal Conditions

Glucural was tested with immortal cell cultures for its ability to protect different eukaryotic cells (mammalian cells and yeast) at various sub-optimal conditions. The following extreme conditions were imposed on the cell cultures: freeze/thaw cycles, contact with detergent (DMSO—a toxic compound, but a very popular cryo-protector), starvation and growth in a saturated state, etc. In all cases, Glucural exhibited excellent cytoprotective action.

I. Growth Activity Following Maximal Cell Damage:

The goal of the study was to test the effect of Glucural on damaged cells growing in very harsh conditions. Mouse erythroleukemia (MEL) cells were damaged from exposure to two freeze/thaw cycles. The cells were then grown in the presence of 10% dimethylsulfoxide (DMSO), a substance known to damage cell membranes. Following that, 10⁷ cells in the late log phase were pelleted and re-suspended in RPMI 1640 medium containing 20% FBS and 10% DMSO and supplemented with Glucural concentrations of 0, 5, 10, 25, and 50 mkg/ml. The cells were then subjected to two freeze/thaw cycles (the cells were quickly brought to −135° C., incubated for 1 hour at −135° C. and quickly thawed to 100% humidity and 37° C.). The cell concentration was then determined by counting, using a hematocytometer. The experiments were done in duplicate.

The results are as follows:

| Glucural, mkg/ml | The number of cells per 1 ml, × 10⁴ |
|---|---|
| 0 | 0 |
| 5 | 0 |
| 10 | 0 |
| 25 | 53 ± 7 |
| 50 | 107 ± 7 |

In extreme conditions, no cell survival was observed without Glucural. Optimal protection from cell damaging was achieved with concentrations of 25 and 50 mkg/ml of Glucural.

II. Growth of Cells (Intact, and after Mild and Moderate Damage) in Optimal Conditions

The goal of this study was to assess the cell toxicity of Glucural on intact cells in optimal growth conditions and to test the influence of Glucural in cell recovery after mild and moderate damage.

Cell toxicity of Glucural in Intact Conditions: Growth of MEL in the presence of Glucural. Intact MEL cells were re-suspended at 10 cells/0.1 ml in RPMI 1640 containing 10% FBS in 5% CO₂, 100% humidity and at 37° C. in the presence of 0, 25, 50, 100, 200, and 400 mkg/ml of Glucural, and were then incubated for 24 hours at 37° C.

Mild Damage: Influence of Glucural on cells damaged by one-freeze/thaw cycle. Cells were treated exactly as above except that they were subjected to one freeze/thaw cycle (in the absence of cryo-protector) prior to incubation at 37° C. for 24 hours.

Moderate Damage: Influence of Glucural on cells damaged by storage for three days at −135° C. in cryo-protector followed by growth for 24 hours in the presence of 1% DMSO. 107 cells were re-suspended in cryo-protector (RPMI 1640 containing 20% FBS and 1% DMSO), supplemented with Glucural concentration of 0, 10, 25, 50, 100, and 200 mkg/ml. The cells were then brought to −135° C. over a two-hour period and stored at this temperature for 3 days. The cells were then thawed quickly by incubation at 37° C., diluted ten times in culture medium containing increasing amounts of Glucural, and incubated for 24 hours at 37° C. Results are noted below. The number of cells per ml. is x 10⁴, columns represent different assays.

| Glucural | Regular | | Moderate |
|---|---|---|---|
| (mkg/ml) | conditions | Mild damage | damage |
| 0 | 70 ± 1 | 16 ± 1 | 59 ± 1 |
| 25 | 67 ± 1 | 36 ± | 58 ± 3 |
| 50 | 68 ± 1 | 53 ± 5 | 76 ± 1 |
| 100 | 71 ± 2 | 40 ± 1 | 82 ± 1 |
| 200 | 58 ± 3 | 45 ± 0 | 163 ± 3 |
| 400 | 69 ± 5 | 31 ± 1 | N.D. |

There is no influence of Glucural from 25 to 400 mkg/ml for regular conditions and cytoprotective effect for cells growing at sub-optimal conditions.

III. Storage at −135° C.

The goal of the study was to assess the effects of Glucural on cell recovery after short-term and long-term storage at −135° C. 10⁷ cells were re-suspended in cryo-protector (RPMI 1640 containing 20% FBS and 10% DMSO), and supplemented with Glucural concentrations of 0, 10, 25, 50 and 200 mkg/ml. The cells were then brought to −135° C. over a two-hour period, and stored at this temperature for 3 days (short term) or six weeks (long term). The cells were then thawed quickly by incubation at 37° C., washed twice with growing medium, re-suspended in 5 ml and incubated at 37° C. for 24 hours. The numbers of cells per ml. is ×10⁴, two different assays.

| Glucural (mkg/ml) | Short-Term Storage | Long-Term Storage |
|---|---|---|
| 0 | 36 ± 3 | 60 ± 2 |
| 10 | 34 ± 4 | 122 ± 8 |
| 25 | 38 ± 2 | 167 ± 3 |
| 50 | 33 ± 2 | 200 ± 12 |
| 100 | 37 ± 2 | 169 ± 15 |
| 200 | 56 ± 5 | 155 ± 14 |

During long-term storage, Glucural increased cell survival rates by over 3 times using the optimal concentration of about 50 mkg/ml. It had little or no effect during short-term storage, most likely because of the small amount of cell decay products present under these conditions.

IV. Storage of Mammalian Cells FRTL-5 and ATT-20 at Low Temperature

The goal of the study was to assess the influence of Glucural on cell recovery of different mammalian cells suspended in various cryo-protectors. A single layer of the rat thyroid-derived cell line FRTL-5 was grown in VPF -12 medium containing 10% calf serum (C.S.) in Petri dishes (10 cm) stored in an environment of 5% CO₂, 100% humidity and at 37° C. Cell coverage on the Petri dish bottom reached 80-90% over 6-8 days. A single-layer cell culture of the cell line from the anterior lobe of the pituitary gland (corticotrophs) of ATT-20 was grown in similar fashion but in D-MEM medium containing 10% C.S.

To assay their viability, FRTL-5 and ATT-20 cells were removed from the Petri dish using a routine method (trypsin treatment), re-suspended in a cryo-protector supplemented with Glucural concentration of 0, 50, 100 and 200 mkg/ml, and then exposed to a low temperature (−135° C.) over 1-3 days. After exposure, cell suspensions were melted quickly in aliquots with cell suspension equal to 0.2 or 0.4 ml per well. The growing period varied depending on conditions of cell storage. All trials were performed in duplicate. Cell viability was estimated by the percentage of the well bottom covered by cells. The results are summarized below.

| | | | | Percentage of |
|---|---|---|---|---|
| | Type of | Cryo-protector, | | well |
| | cells, | Storage | Glucural | bottom covered |
| # | Growing time | temperature | mg/ml | by cells |
| 1 | FRTL-5 | 10% Glycerol, | 0 | 60-70 |
| | (4 days) | −70° C. | 100 | 90-100 |
| | | | 200 | 90-100 |
| 2 | FRTL-5 | 10% Glycerol, | 0 | 2-5 |
| | (36 days) | −190° C. | 100 | 10-15 |
| 3 | FRTL-5 | 50% C.S.; 0° C., | 0 | 25-30 |
| | (4 days) | 30 min | 100 | 60-70 |
| | | | 200 | 60-70 |
| 4 | AtT-20 | 10% Glycerol, | 0 | 50-60 |
| | (3 days) | −190° C. | 50 | 50-60 |
| | | | 100 | 80-90 |
| 5 | AtT-20 | 10% Glycerol, | 0 | 5-10 |
| | (44 days) | −70° C. | 100 | 40-50 |
| | | | 200 | 30-40 |
| 6 | AtT-20 | 10% Glycerol, | 0 | 2-5 |
| | (46 days) | −70° C., | 100 | 20-30 |
| | | additional | 200 | 10-20 |
| | | melting | | |
| | | freezing | | |

The same method was used here as in tests with MEL cells. Cryo-protectors used were not optimal for FRTL-5 and ATT-20 cells; Conditions of freezing were not optimal in assays # 1, 5 and 6. Experiments under these sub-optimal conditions demonstrate the protective effect of Glucural for different mammalian cells in sub-optimal conditions.

V. Additional Experiments for the Growth of Immortal Human Monocytes (THP-1 Cells) in Sub-Optimal Conditions

The short-term growth of immortal human monocytes (THP-1 cells) was tested in the presence of 10% DMSO with different dosages of Glucural. Percentage of dead cells (trypan-blue test) and concentrations of live cells (×1,000,000 cells/ml) were determined. The growth medium was RPMI 1640 with fetal serum and other supplements.

10% DMSO plus Glucural (mkg/ml)

| Growing | Control | | | | |
|---|---|---|---|---|---|
| Time | group | 0 | 10 | 20 | 50 |
| | 6.1 ± 0.05% | 29.05 ± 1.85% | 13.3 ± 1.3% | 2.6 ± 2.0% | 17.2 ± 0.4% |
| 4.5 hrs | 2.77 ± 0.3 | 0.91 ± 0.04 | 1.06 ± 0.08 | 0.52 ± 0.05 | 0.65 ± 0.03 |
| | 16.1 ± 0% | 63.3 ± 2.0% | 40.5 ± 0.5% | 36.1 ± 0.75% | 39.5 ± 1.1% |
| 24 hrs | 1.95 ± 0.1 | 0.86 ± 0.04 | 2.21 ± 0.26 | 0.86 ± 0.04 | 1.31 ± 0.02 |

Long-term growth of THP-1 cells in the presence of different levels of Glucural was determined based on the percentage of dead cells (trypan blue test). See below. Concentration of Glucural, mkg/ml. This data indicates that Glucural provides cyto-protective action during long term growing conditions without a change in the medium (starvation).

| Days of growing | 0 | 10 | 20 |
|---|---|---|---|
| 3 | 5.1 ± 0.2 | 2.1 ± 0.5 | 5.5 ± 0.25 |
| 4 | 15.0 ± 2.6 | 7.2 ± 1.3 | 0 |
| 10 | 32.0 ± 4 | 10.7 ± 0.2 | 10.85 ± 0.35 |
| 14 | 29.8 ± 2.1 | 15.3 ± 0.2 | 15.3 ± 0.5 |

The percentage of dead THP-1 cells resulting from DMSO shock was tested, with and without Glucural. Cells were incubated in the presence of 10% DMSO at 37° C. for 90 minutes (tryptan-blue test). Glucural was added at a concentration of 25 mkg/ml.

| | # of assay | | | |
|---|---|---|---|---|
| | 1 | 2 | 3 | 4 |
| Control | 3.6 ± 1.3 | 6.1 ± 2.4 | 4.6 ± 1.2 | 3.4 ± 1.4 |
| Control + | 3.9 ± 0.7 | 5.8 ± 3.1 | N.I. | 3.0 ± 1.0 |
| Glucural | | | | |
| DMSOshock | 12.9 ± 6.2 | 18.2 ± 5.3 | 17.9 ± 9.9 | 8.8 ± 5.2 |
| DMSOshock + | 4.1 ± 1.6 | 7.7 ± 3.4 | 4.6 ± 2.2 | 5.0 ± 2.3 |
| Glucural | | | | |

The above data indicates that Glucural provides cyto-protective action during long-term growing conditions without a change in the medium (starvation) and “DMSO shock”. At the same time, there is no influence of Glucural on cell growing for Control and Control+Glucural groups.

Longer-duration studies were conducted to determine Glucural's ability to protect cells during starvation and “DMSO Shock”. Four assays were conducted (4-6 day duration), each with group A (control), B (control plus Glucural), C (DMSO shock) and D (DMSO shock with Glucural). Growth was measured after the administration of “DMSO Shock” and without a change of medium. Glucural concentrations of 25 mkg/ml were used and there was no change in the medium. Trypan-blue test was used: the number of live cells per 0.1 ml (based on 4 counting iterations).

| | # of assay | | | |
|---|---|---|---|---|
| | #1 | #2 | #3 | #4 |
| | days growing | | | |
| | 5 days | 6 days | 4 days | 4 days |
| A Control | 272 ± 29 | 193 ± 36 | 142 ± 12 | 161 ± 14 |
| B Control + Glucural | 241 ± 24 | N.I. | 136 ± 5 | 160 ± 22 |
| C DMSO shock | 172 ± 30 | 145 ± 22 | 110 ± 5 | 139 ± 17 |
| D DMSO shock + | 228 ± 48 | 184 ± 19 | 137 ± 6 | 222 ± 42 |
| Glucural | | | | |

The optimal dose of Glucural for THP-1 cells is 25 mkg/ml. Glucural demonstrated protective effect in the event of “DMSO shock” and starvation and absence of any influence for Control+Glucural group.

Primer Cell Cultures

One particularly attractive application of the cytoprotective quality of 6-methyluracil compounds is to extend the life span of primer cell cultures. Primer cell cultures are cell cultures obtained from various tissues. They are used routinely for new drug screening in pharmaceutical industry. The problem associated with many primer cell cultures is their general inability to propagate and grow, leading to their early death and therefore limiting their use in drug research. For example, a rat embryo brain neuron cell culture has about 80% mortality in 12-14 days. Application of Glucural allowed for increasing the life span of these cells by more than two fold.

The following is an example of the difficulty in maintaining the survival rate of primer cell cultures. The primer cell cultures of brain neurons and liver cells, among others, do not multiply or replicate themselves. The term “growing” of these primer cell cultures means maintaining their survival for a 10-15 day period. But these cell cultures are very valuable as test objects and are commonly used for the initial testing of new drugs (drug screening). Glucural was tested on the neurons from rat embryos brains that were transferred into cell cultures. The viability of these neurons was tested during 2 stages of the production of embryonic neuron cell cultures: 1) mechanical disassembly and washing, and 2) the “growing” of the cell culture. It was demonstrated that Glucural slowed cell death during both of these stages very effectively.

Glucural was also tested on two primer cell cultures for protection against cell death in sub-optimal conditions. The cytoprotective capability of Glucural on human endothelial cells from human umbilical cord vein was tested in several typical conditions causing cell death: starvation (growth without changing medium), mechanical damage, enzymatic damage and exposure to the natural inducers of mammalian cell death, human tumor necrosis factor-alpha (TNF-alpha).

Human umbilical cord vein endothelial cells (HUCVEC) were isolated from freshly obtained human umbilical cords in accordance with known methods. HUCVEC were grown in multi-well plates. After saturation of the wells, the assays were initiated. Four wells were used for each experimental group. To cause “mechanical” damage, a higher (2-2% times) centrifugal speed was used to pick up cells after trypsin treatment. To cause enzymatic damage, full-strength trypsin solution was used instead of the recommended diluted concentration. All assays were started from the last change of medium in wells with saturated cell culture (well bottoms were covered completely by cells). Results were estimated from the percentage of the bottom covered by cells on the last day of the assay.

Assay 1. Enzymatic and mechanical damage to cells: Growth after 7 days, without change of medium.

| Glucural, mkg/ml | Percent of well-bottom covered by cells |
|---|---|
| 0 | 0 |
| 40 | 2-5 |
| 400 | 70-80 |

Assay 2 and 3. Starvation—cell growth without change of medium during 11 days. Assays 2 and 3 were performed using two different HUCVEC isolations.

Percent of well-bottoms covered by cells

| Glucural, mkg/ml | Assay 2 | Assay 3 |
|---|---|---|
| 0 | 0 | 20-40 |
| 300 | 70-80 | 100 |
| 400 | 80-90 | 100 |
| 500 | 80-90 | 100 |

To test for cell damage, HUCVEC were exposed for period of 6 to 24 hours to the human TNF-alpha. The trypan-blue test was used. After exposure, mono-layers were removed by trypsin treatment. Results were estimated based on the percentage of colored dead cells in the mono-layer.

Assay 4. HUCVEC exposed for 24 hours to TNF-alpha and Glucural.

| | | Percent of dead cells |
|---|---|---|
| TNF-alpha, ng/ml | Glucural mkg/ml | in mono-layer |
| — | — | 12.2 ± 0.9 |
| — | 500 | 4.4 ± 0.4 |
| 20 | — | 20.45 ± 1.6 |
| 20 | 500 | 10.3 ± 1.3 |

Assay 5. HUCVEC exposed for 8 hours to TNF-alpha and Glucural.

| | | Percent of dead cells |
|---|---|---|
| TNF-alpha, ng/ml | Glucural, mkg/ml | in mono-layer |
| — | — | 8.8 ± 0.2 |
| — | 500 | 5.7 ± 0.5 |
| 10 | — | 10.35 ± 0.05 |
| 10 | 500 | 4.1 ± 0.4 |
| 20 | — | 14.85 ± 0.15 |
| 20 | 500 | 5.4 ± 0.4 |

Assay 6. HUCVEC exposed for 6 hours to TNF-alpha and Glucural.

| | | Percent of dead cells |
|---|---|---|
| TNF-alpha, ng/ml | Glucural, mkg/ml | in mono-layer |
| — | — | 23.9 ± 9.8 |
| — | 500 | 13.8 ± 2.7 |
| 40 | — | 30.3 ± 4.7 |
| 40 | 500 | 10.9 ± 3.4 |

As demonstrated in the examples above, Glucural provides significant cytoprotection capability for primer cell cultures at all sub-optimal conditions.

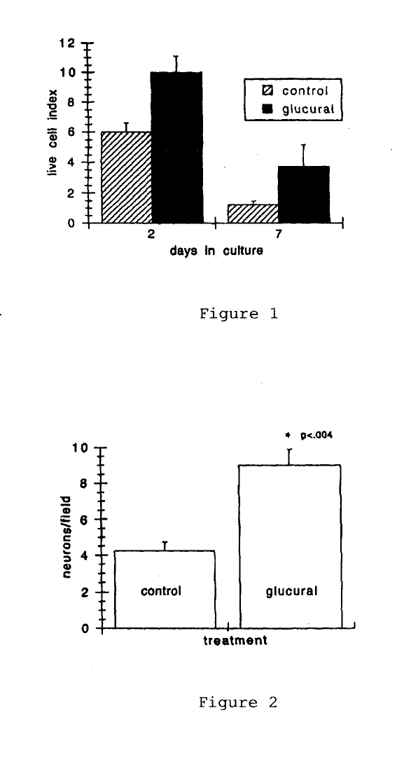

Another study was conducted on the effect of Glucural on cell survival after mechanical damage. The brains of rat embryos were mechanically disassociated in the presence of as well as absence of 500 mkg/ml of Glucural and plated, and the medium was changed after several hours. After one day, the number of live neurons was found to be more than double in the culture that included Glucural. After 7 days, this ratio increased to nearly triple as shown on FIGS. 1 and 2.

Organ Transplants

Glucural can be used to improve the viability of organ transplants, especially when there is a significant time period between organ removal from the donor patient (or cadaver) and implantation into the receiving patient. Glucural has been determined to suppress cell death in human endothelial cells, and this is thought to occur based on its cytoprotective action in cell cultures. Endothelial cells may be damaged by various diseases and require protection for repairing and restoring of their function.

In the area of organ transplantation, it is well known that destruction of endothelial cells is a lead factor in the process of organ damage. It is known that the cutting of vessels supplying blood to endothelial cells signals the production (or release) of tumor necrosis factor-alpha (TNF-α). This substance is thought to be a primary signaling molecule triggering the apoptosis process of the transplanted organ cells after entering through the cell membranes. Glucural suppresses the effect of TNF-α to initiate apoptosis and therefore protects all organs from TNF-α action. Endothelial cells of blood vessels are damaged in the case of all metabolic dysfunctions. Their positive state is very important for the success of organ transplantation. The use of the compounds of the invention is believed to prevent such cell damage and extend the time available for organ preservation and transplantation. Glucural may be administered intravenously to the donor before organ removal and also can be applied to the organ during its storage.

Immortal Mammalian Cell Cultures

The compounds of the invention can also be used for cytoprotection of immortal mammalian cell cultures that are widely used in many branches of biotechnology, cell biology research and in many commercial processes. They can multiply endlessly. For comparison, primer cell cultures obtained from regular healthy tissues are restricted in their number of cell divisions (typically less than 40 divisions), or they do not divide at all. People are interested in the safe storage of inoculum (cells stocks in storage, which are used in different cell lines, fermentations, commercial production processes, etc.). Many of these cell cultures exhibit low viability under various states of storage, such as, after transformation, lyophilization or during storage in the frozen state. The compounds of the invention were found to be useful in preventing cell damage and improving cell viability in all of these conditions.

Yeast Industry

Yeast cells of various strains are widely used in the food industry, as well as in biotechnology and bioresearch. Unlike other eukaryotic cells, yeast cells can be lyophilized (freeze-dried) for storage. Lyophilization is a common method for the preparation and storage of yeast inoculums. However, depending on the strain, this process can decrease cell viability by as much as 30, 40, or in some cases over 99%. The compounds of the invention were found to significantly increase the viability of yeast cell cultures after lyophilization. It will be particularly useful to apply the method of the invention to the weak strains of these cell cultures, as they are more vulnerable during lyophilization. The use of Glucural will allow the use of a much smaller amount of yeast in the preparation of inoculums of the best quality.

The lyophilization of yeast is a very common technique used in different fields of industry, biotechnology and research. The viability of lyophilized yeast species and strains can vary greatly. The viability of commercial baking yeast after lyophilization is high (50-80%). But other strains of yeast used in industry and research, can have viability as low as 0.001-0.001%. Depending on the yeast strain, viability after lyophilization using Glucural can be increased by 2 to 100 fold. Glucural was tested using the strain of yeast called Saccaramices Cerevisiae, a strain with a very low level of viability after lyophilization. By increasing the viability of lyophilized yeast inoculums, the amount of yeast required to make such inoculums may be lowered.

The optimal dose for Glucural to protect yeast cells was found to be 25 mkg/ml, added prior to lyophilization. The standard method of lyophilization was used. The strain of baking yeast tested was grown in standard conditions and re-suspended in Glucural at a concentration of 6.6×10⁹ cells/ml. The suspension (1 part) was added to the Glucural solution (9 parts) to produce the final volume. The suspensions were aliquoted and lyophilized at standard conditions.

To assay the vitality of lyophilized yeast, two approaches were used:

The lyophilized yeast from an ampoule was re-suspended in 1 ml of regular growing medium and diluted by 10, 100, and 1,000 times. The suspensions of yeast were plated on the regular YPD agarose medium by 20 mkl per plate. After growing the colonies for 48 hours at 28° C., the number of colonies was counted.

This test included growing lyophilized yeast in 10 ml of growth medium for 24 hours, before plating on the agarose plates as described above.

Three lyophilizations with 3-4 trials each for both approaches demonstrated similar results. The viability of the yeast strain tested increased by a factor of between 50 to 100 compared to the control group.

Additionally, some industrial processes such as fermentation involve sub-optimal growing conditions (non-optimal temperature) for yeast cell cultures, causing apoptosis. The compounds of the invention may be used with positive results in the protection of cell cultures from damage during these circumstances, such as in the brewing of beer, baking bread, etc.

Medical Uses of Glucural:

Glucural can be used as a substitute for thalidomides in the treatment of different degenerative diseases such as Parkinson's, Alzheimer's, etc. Glucural was found to have equal anti-inflammatory action as thalidomide during standard tests, as performed by Dr. Kirk Sperber of the Mount Sinai School of Medicine (unpublished data). The water-solubility of Glucural is more than ten times higher than thalidomide (water-solubility is a limiting factor in the use of thalidomide for many applications in clinical practices). Glucural can also be used for the regeneration of skeletal muscle cells (trauma), heart muscle cells (myocardial infarction), liver cells and kidney cells.

The need for a medical application of a compound capable of reducing the excessive or inappropriate apoptosis is described in detail in the U.S. Pat. No. 6,403,792 which is incorporated herein in its entirety. It has been shown that inhibition of apoptosis may be a novel therapy for the treatment of the following diseases: Ischemia/reperfusion, viral infections, stroke, polycystic kidney disease, glomerulo-nephritis, osteoporosis, various types of anemia, chronic liver degeneration, T-cell death, osteoarthritis, male pattern baldness, Alzheimer's and Parkinson's, and type I diabetes. Application of 6-methyluracil based compounds and Glucural in particular presented in pharmaceutically acceptable forms and solutions to treat these conditions is suggested in the present invention.

Another particularly attractive use of the compound of the invention is to increase the viability of cells in various cloning techniques, such as for stem cells and transfer of nuclei for example. Cells exposed to Glucural in the above mentioned concentrations would exhibit increased survival and viability.

A further particularly attractive use of Glucural is in the area of cosmetics. The general need in cosmetics is well described in the U.S. Pat. No. 6,355,280 by Segal, which is incorporated herein in its entirety. Apoptosis is a major contributor to skin cell damage. Skin is subjected daily to a variety of sub-optimal conditions such as environmental factors and pollutants. Cosmetic compositions containing 6-methyluracil based compounds are believed to be able to reduce skin cell damage and protect the skin from such negative conditions. Uses of Glucural in Food Preservation:

In line with its ability to inhibit the leakage of lysosomal enzymes, the compounds of the invention were found to decrease proteolytic tissue decay. Proteolytic tissue decay has important implications in the food industry, in particular in the preservation of all meat, including red meat, pork, fowl, fish, etc. Food preservation methods typically aim to slow the process of autholysis, or the self-digestion of cell tissue, and multiplication of microbes. Various techniques are used for such preservation, including salting, freezing, canning, and preventing oxidation by using inert gases or antioxidants, etc. However, there are no known methods available for the protection of tissue from autholysis itself.

The compounds of the present invention demonstrated excellent properties in significantly slowing or even arresting the autholysis process. Generally, this process depends on the stability of lysosome membranes. Glucural increases this stability, preventing or slowing autholysis.

Glucural has other attributes that make it attractive as a food supplement. Glucural is low in toxicity. Its LD₅₀ is about 2 g/kg for mice, or the equivalent of 100 g for a 50 kg person. Glucural is a small compound that is relatively easy to digest when taken orally. Glucural is inexpensive to manufacture and its shelf life is more than 10 years at ambient temperatures.

Preservation of Fish

Applying a water solution of the compounds of the present invention to fish soon after they have been caught will slow or arrest their proteolytic decay. Presently, the initial preservation of fish is done on the fishing boat, where the fish is often washed in a salt solution and maintained at low temperature with ice or refrigeration. Application of the compounds of the present invention will allow for storing this fish at higher (or even ambient) temperatures, or for a longer time at sea. This is true for all fish, whether it will later be sold as fresh fish, canned, salted, processed, etc.

Fish that is preserved using the salting method will maintain its quality (low level of proteolytic decay) longer with the application of Glucural prior to salting. Fish and caviar that are preserved in cans or jars will have their shelf life extended when treated with Glucural (added to liquid in the can or jar with fish or caviar). Frozen fish may be stored longer and better maintain their good quality if treated with Glucural prior to being frozen. Here, “dry salting” can be used with adding concentrated solution of Glucural in which no water is used to deliver the additive.

Salted fish, generally herring and salmon, are produced and consumed in large quantities in many countries of the world. The methods of fish salting, however, have not changed much since ancient times. For over 4,000 years, fishermen have known that herring cannot be salted during the “season of intensive feeding” because the herring will “over mature” quickly, making the fish caught during this season unsuitable for this preservation method. For salted fish, the storage duration (and storage temperature) permissible is limited because it will over-mature (too much proteolytic decay). This situation seasonally increases the price of this product.

We propose a new method to improve the shelf life of salted fish. To slow the proteolytic decay that causes the maturation (and over-maturation) of salted fish, Glucural was tested. Very small amounts of Glucural (less than 100 mg per kg of fish) were added to the salting solution and the rate of maturation of the salted fish was greatly reduced.

Baltic sprats (small herring) caught during the season of intensive feeding were tested. Three different catches of Baltic sprats were used during these trials. The fish was salted with the “dry method”: NaCl was added to the fish in an amount equal to 7.5% of their weight. Glucural was added as a water solution. The standard cans of fish were hermetically sealed and kept at 6 degrees C. for 7.5 months. The normal duration of fish storage is two months, while 4 months of storage usually results in “over-maturing”. This storage time applies only to fish not caught during the season of intensive feeding. The fish tested with Glucural was caught during the season of intensive feeding, so the salt maturation period is usually much shorter.

General methods used in the preservation of fish with Glucural for the salting method were as follows. Produce a 1% solution of Glucural and water (10 mg of Glucural per 1 ml of water). Agitate the water with Glucural until the Glucural goes into solution. Glucural is water soluble, so it should take less than 10 seconds to go into solution. For small volumes, manual shaking can be used. For larger volumes, a magnetic bar or other mixing mechanisms can be used. For each kilogram of fish (small herrings) to be salted, add 2 ml of the 1% Glucural solution to the brine to be used. This means that 20 mg of Glucural is sufficient for treating 1 kilogram of fish.

A panel consisting of 5 expert tasters performed the standard tests of the salted fish. The “NaOH degree” test was also performed, by which the amount of —COOH groups in the fish tissue was evaluated. The presence of these groups serves as an indication of proteolytic decay according to official test methods. In our tests, we applied 5 different concentrations of Glucural. Of these different concentrations, one retarded maturation to 5 months, while another delayed it by 7.5 months. A control group (without Glucural) was completely over-matured and totally unusable after 3.5 months. The range of Glucural concentrations tested was between 5 and 100 mg/kg of fish. Additional tests of Glucural were performed to extend the period of storage of horse mackeral, and similar results were obtained.

These tests indicate that using Glucural will improve the effectiveness of the preservation of fish using the salting method and expand the types of fish that can be preserved in this manner. The optimal dose of Glucural required for this purpose is low in cost, projected at less than 3% of the final cost of the product. Optimal concentrations of Glucural can vary greatly, from 1 mg to 1 g of Glucural for each kilogram of fish, depending on the fish species.

The use of Glucural in the salting of fish will allow the following:

The salting of various species of herring during the season of intense feeding. Previously, this was not considered possible.

Salted fish can be stored longer and/or at higher temperatures.

Possibly increase the number of fish species that are eligible for salting.

Reducing the Autolysis of Mammalian Muscle Cells Using Glucural

Preliminary methodological research indicates that the autolysis (self-digestion) of tissue after the death of an animal is caused largely by the release of lysosomal proteases. According to our data, the leakage of lysosomal enzymes can be decreased under the action of the appropriate dosage of Glucural. This indicates that Glucural is capable of arresting autolysis in animal flesh, thus better maintaining the quality of meat during storage.

For the refrigerated (or frozen) storage of beef, pork, fowl, etc., particularly in the range of 0 to 6° C., Glucural will slow the progress of proteolytic decay of the meat tissue. This slowing of proteolytic decay will extend the time that the meat can safely be stored prior to consumption. It will also allow an increase in the meat's refrigeration temperature, while still maintaining its quality for consumption. This is especially true for meat that is ground up (sausages, ground meat, processed meat, etc.), due to its faster rate of proteolytic decay. The animal can ingest Glucural a day before its slaughter, or preferentially, it can be injected intravenously (quantity based on the weight of animal) several (2-4) hours prior to slaughter. Also, treating the outer surface of meat (and in particular, ground meat) with a liquid solution containing the appropriate dosage of Glucural will delay proteolytic decay.

In-vitro Tests

In-vitro tests were performed on the skeletal (rear leg) muscles of 17 white laboratory rats. Preliminary research efforts determined 1) the preparation method for muscle homogenates, 2) estimation of level of the proteolytic activity by measuring for the concentration of free amino acids, a result of protein cleavage, and 3) the optimal time of authoproteolysis.

The preparation of muscle homogenates. Efforts were made to disassemble the muscle tissue gently, without disruption of the lysosomes, though with sufficient strength to ensure a homogeneous suspension of muscle cells in physiological solution (PBS). Potter's homogenator was used with a glass barrel and a Teflon pestle. Determination of the optimal treatment was based on experimental data and the activity of the proteases during the incubation of the homogenates at 37° C. If lysosomes were damaged during preparation of the homogenates, then there was no increase in concentration of free amino acids during incubation. Undamaged lysosomes in the homogenate would continue their proteolytic activity during incubation. For the best preparation of the homogenate, the muscles were separated from the connecting tissue, weighed and cut up with scissors in a Petri dish for 10 minutes. The tissue was transferred into the barrel of the homogenator and cold, standard PBS was added ten parts to one part of tissue (weight to volume). The tissues were turned into homogenate with 6 strokes of the pestle. Glucural was dissolved in water (2.5 mg/ml) and added to the PBS before the treatment. All manipulations were done at 0° C.

The estimation of proteolytic activity. After incubation of the muscle samples for 4 hours, they were iced and then centrifuged for 10 minutes at low speed. An incubation time of 4 hours at 37° C. was chosen as optimal because a longer time, such as 24 or 48 hours, did not allow the generation of good data due to lack of stability of the lysosome membranes past 4 hours.

After the incubation of samples, the protein concentration was estimated by the standard Bio-Rad method. To estimate proteolytic activity based on the concentration of free amino acids found in the sample, three methods were tried: 1) absorbance at 280 nm, 2) concentration of amino acid tyrosine by estimation with Folin reagent, and 3) concentration of end amino groups with Ninhidrin reagent. Only method 3 provided accurate results, so the proteolytic index was measured based on the Ninhidrin reagent. After the estimation of protein concentrations, we used aliquots 0.05 or 0.1 ml, added water to have the volume equal to 0.8 ml and added 0.8 ml of 0.1% Ninhidrin water solution. Samples were boiled in a water bath for 10 minutes, chilled and the absorbance at 560 nm was measured. The concentration of free ends of NH₂ groups was estimated by the curve, which was done with amino acid tyrosine. Concentrations of free amino acids and protein in mg/ml could therefore be determined for both cases. The ratio of free amino acids per proteins in the sample was calculated as the proteolytic index. For each time point, 5 rats were used. The estimation of the “proteolytic index” for “control” samples (without incubation) was performed to provide a base point. Each homogenate was obtained from the muscles of one rat, n=5 (5 rats per assay).

Action of Glucural on Proteolytic Index of Muscle Homogenates, mg of aminoacids per mg of protein.

Time of Incubation of 4 Hours and temperature at 37° C.

| # | Glucural, mkg/ml | Proteolytic Index | P value |
|---|---|---|---|
| control | — | 1.100 ± 0.113 | — |
| 1 | — | 2.172 ± 0.323 | <0.01 vs. control |
| 2 | 20 | 2.142 ± 0.274 | <0.05 vs. 1 |
| 3 | 30 | 2.268 ± 0.195 | >0.05 vs. 1 |
| 4 | 50 | 1.380 ± 0.170 | <0.05 vs. 1 |
| 5 | 100 | 1.071 ± 0.116 | <0.01 vs. 1 |

Conclusions

Glucural at concentrations of 50 and 100 mkg/ml decreased proteolytic activity in muscles of 36% and 51%, respectively (Group numbers 4 and 5).

Concentrations of Glucural of less than 50 mkg/ml are not sufficient to reduce autoproteolysis.

Radiation Protective Action of Glucural and Stabilization of Genome:

The suppression of proteolytic activation caused by Glucural suppresses the mutagenic action of radiation on cells. Glucural can be used to prevent, and treat, the cell damage caused by exposure to radiation (X-rays, etc.). There is no evidence of any radiation protective qualities of other commonly known anti-inflammatory drugs, only the suppression of free radicals. Also, Glucural will stabilize the genome of different biological matter, and this can be used for many different purposes, including in clinical practice.

The use of Glucural was investigated as a radio-protector to decrease the mutagenic action of irradiation. The classic genetic model was used, based on the well-known method: the frequency of mutations in Drosophila (fruit flies) caused by X-rays. It is known that the frequency of these mutations can be increased by adding to the food of females certain membrane destabilizers (we used Polyene Antibiotics—Amphotericin B), or by the use of a special diet. Like all insects, Drosophila obtain all of their sterols from food (yeast for Drosophila). A special diet rich with Nistatin (Polyene antibiotic) resistant yeast was used.

The frequency of the loss of x-chromosomes (males excluded) was measured after crossing with the females. Females were given a dose of 1000 Roentgens 5 days before being given the food additive or “special diet”. After the mating, the percent of exclusive males demonstrated the mutations frequency of oocytes in females.

The data obtained from these tests confirms the critical importance of the state of the nuclear proteins for DNA accessibility, as follows: Glucural demonstrated the ability to prevent the damaging action of irradiation; Glucural can be used as a new anti-mutagenic compound for research and for different clinical applications.

The mechanism of Glucural anti-mutagenic and radio-protective actions is different from other known chemicals. We assume that it can be used in combination with other known radio-protective and, anti-mutagenic substances to obtain a synergistic effect. The following results illustrate the point above:

Influence of drugs for X-Ray caused loss of X-chromosomes
in late oocyte of Drosophila (% of exclusive males):

| | | | | | P |
|---|---|---|---|---|---|
| | | Amount of | | | #3-6 |
| | | descendants | | Frequency | relatively #1 |
| Experimental | | Regular | Exclusive | of the loss of | #8-11 |
| group | | females | males | X-chromosomes, % | relatively #7 |
| 1. | control | 2174 | 34 | 1.5 ± 0.26 | >0.05 |

Glucural, mkg/female

| | | | | | |
|---|---|---|---|---|---|
| 2. | 1.0 | 3585 | 63 | 1.7 ± 0.21 | |
| 3. | 2.0 | 2644 | 30 | 1.1 ± 0.20 | <0.05 |
| 4. | 2.5 | 2738 | 26 | 0.9 ± 0.18 | <0.01 |
| 5. | 5.0 | 3656 | 27 | 0.7 ± 0.14 | <0.001 |
| 6. | 10.0 | 3121 | 39 | 1.2 ± 0.19 | <0.05 |

Amphotericin B, mkg/female

| | | | | | |
|---|---|---|---|---|---|
| 7. | 1.0 | 3587 | 52 | 1.4 ± 0.19 | |
| 8. | 5.0 | 4546 | 98 | 2.1 ± 0.21 | <0.05 |
| 9. | 10.0 | 2681 | 100 | 3.6 ± 0.35 | <0.001 |
| 10. | 15.0 | 2278 | 97 | 4.1 ± 0.41 | <0.001 |
| 11. | 20.0 | 1038 | 45 | 4.2 ± 0.61 | <0.001 |

Influence of sterol-deficit diet and both drugs on the frequency of X-chromosomes loss in late oocytes of Drosophila. Groups 1-4 received yeast of wild type, groups 5,6 (diet) received nistatin resistant yeasts meaning groups 5,6 had abnormal sterols in membranes:

| | Amount of | | | | |
|---|---|---|---|---|---|
| | Descendants | | Frequency of | | Protective |
| 1. Experimental | Regular | Exclusive | X-Chromosomes | | Effect |
| Group | females | Males | loss, % | P Value | of Glucural, % |
| 2. Control | 3777 | 69 | 1.8 ± 0.21 | | 50 |
| 3. Glucural | 4287 | 39 | 0.9 ± 0.14 | <0.001 | |
| 4. Amphotericin B | 4575 | 206 | 4.3 ± 0.29 | | 26 |
| 5. Amphotericin | 2553 | 84 | 3.2 ± 0.34 | <0.05 | |
| B/Glucural | | | | | |
| 6. Diet | 2611 | 155 | 5.6 ± 0.44 | | 27 |
| 7. Diet /Glucural | 3503 | 150 | 4.1 ± 0.33 | <0.01 | |

Method of Producing of the Compounds of the Invention

The present invention also provides for several novel

methods of producing the compounds of the invention. The prior art, U.S. Pat. No. 3,912,714 by Kulbach, describes one known technique of producing a similar compound, namely 4-methyluracil based substance. This patent is incorporated herein in its entirety by reference. Briefly, N-methyl-D-glucosamine complex of 6-methyluracil is produced by reacting 6-methyluracil with an equimolar amount of N-methyl-D-glucosamine in an aqueous medium at a temperature of 20-50° C. and isolating the product.

According to the method of the invention, Glucural is produced by dissolving 2,4-dihydroxy-6-methylpyrimidine in aqueous solution of equimolar amount of N-methyl-d-glucamine at a temperature from about 60 to about 70° C., and isolating the Glucural product from the solution. Specific examples are as follows:

### EXAMPLE 1

According to the present invention, Glucural is produced by dissolution of 2,4-dihydroxy-6-methylpyrimidine in aqueous solution of equimolar amount of N-methyl-D-glucamine at a temperature of about 60 to 70° C., preferably about 65° C. The resultant transparent colorless or slightly yellowish solution was concentrated under diminished pressure at a bath temperature 45±5° C. to the white or slightly yellowish slurry which was coevaporated with 2-propanol under the same conditions yielding white or slightly yellowish solid residue. This residue after air-drying at ambient temperature in ventilated space produced Glucural with a quantitative yield.

Glucural comprises a white or slightly yellowish water-soluble solid powder with very weak peculiar odor that is faint.

### EXAMPLE 2

The mixture of 2,4-dihydroxy-6-methylpyrimidine (12.61-15.76 g; 100-125 mmol), N-methyl-D-glucamine (19.52-24.40 g; 100-125 mmol), and water (250-315 ml) was stirred at 65+5° C. to the white or slightly yellowish slurry which was co-evaporated with 2-propanol (200-250 ml) yielding white or slightly yellowish precipitate. The later after air-drying at ambient temperature in a ventilated space produced Glucural with practically quantitative yield (31.94-40.16 g; 99.4-99.9%). The Glucural comprises a white or slightly yellowish water-soluble solid powder with very faint peculiar odor.

In another alternative embodiment, the Glucural product may be isolated by crystallization. The solvents used for crystallization or isolation may be also selected from the group consisting of water, propanol, ethanol and isopropanol. The method of making Glucural has low amount of waste and solvents used in the method are recoverable. The method of making the complex with propanol is advantageously environmentally friendly and safe.

Although the present invention is described for specific compounds and their applications, it is not limited thereto. Numerous other variations of the practical applications and modifications of the chemical compounds would be readily appreciated by those skilled in the art and are intended to be included in the scope of the invention. The scope is limited therefore only by the appended claims as follows.

What I claim is as follows:

1. A composition for prevention of lysosomal leakage in eukaryotic cells comprising a 6-methyluracil based water-soluble compound in concentration from about 1 to about 1000 micrograms per milliliter of a biocompatible water solution.

2. The composition as in claim 1, wherein the concentration of said 6-methyluracil compound is from about 10 to about 500 microgram per milliliter of said water solution.

3. The composition as in claim 1, wherein said 6-methyluracil compound is a complex of 2,4-dihydroxy-6-methylpyrimidine with N-methyl-D-glucamine.

4. A method for prevention of lysosomal leakage in eukaryotic cells including a step of exposing said cells to a 6-methyluracil based water-soluble compound.

5. The method as in claim 4, wherein said 6-methyluracil compound is dissolved in a biocompatible water solution.

6. The method as in claim 5, wherein the concentration of said 6-methyluracil compound is from about 1 to about 1000 microgram per milliliter of said water solution.

7. The method as in claim 6, wherein said concentration is from about 10 to about 500 micrograms per milliliter.

8. The method as in claim 4, wherein said 6-methyluracil compound is a complex of 2,4-dihydroxy-6-methylpyrimidine with N-methyl-D-glucamine.

9. A method of providing cytoprotection by prevention of lysosomal leakage in eukaryotic cells exposed to a sub-optimal condition including a step of exposing said cells to a 6-methyluracil based water-soluble compound prior or during said exposure.

10. The method as in claim 9, wherein said 6-methyluracil compound is a complex of 2,4-dihydroxy-6-methylpyrimidine with N-methyl-D-glucamine dissolved in a biocompatible water solution in concentration of about 10 to about 500 micrograms per milliliter.

11. A method for improving eukaryotic cell survival during long-term cryogenic storage including a step of exposing said cells to a composition comprising a 6-methyluracil based water soluble compound prior, during, and after cryo-suspension, said composition dissolved in a biocompatible water solution in a concentration from about 10 to about 500 micrograms per milliliter.

12. The method as in claim 11, wherein said 6-methyluracil compound is a complex of 2,4-dihydroxy-6-methylpyrimidine with N-methyl-D-glucamine and said concentration is about 50 micrograms per milliliter.

13. A method for extending a life span of primer cell cultures including exposure thereof to a 6-methyluracil based water soluble compound dissolved in a biocompatible water solution in concentration from about 1 to about 1000 micrograms per milliliter.

14. The method as in claim 13, wherein said compound is a complex of 2,4-dihydroxy-6-methylpyrimidine with N-methyl-D-glucamine and said concentration is from about 10 to about 500 micrograms per milliliter.

15. A method of preservation of a donor organ for organ transplantation including a step of exposing said organ to a 6-methyluracil based water-soluble compound dissolved in a biocompatible water solution in concentration from about 1 to about 1000 micrograms per milliliter.

16. The method as in claim 15, wherein said compound is a complex of 2,4-dihydroxy-6-methylpyrimidine with N-methyl-D-glucamine and said concentration is from about 10 to about 500 micrograms per milliliter.

17. A method of yeast lyophilization including exposure of said yeast prior to lyophilization to a 6-methyluracil based water-soluble compound dissolved in a biocompatible water solution in concentration from about 1 to about 1000 micrograms per milliliter.

18. The method as in claim 17, wherein said compound is a complex of 2,4-dihydroxy-6-methylpyrimidine with N-methyl-D-glucamine and said concentration is from about 10 to about 500 micrograms per milliliter.

19. The method as in claim 18, wherein said concentration is about 25 micrograms per milliliter.

20. A pharmaceutical composition for treatment of excessive or inappropriate apoptosis, said composition comprising a 6-methyluracil based water-soluble compound and a pharmaceutically acceptable carrier or diluent, said compound dissolved in said carrier in concentration from about 1 to about 1000 micrograms per milliliter.

21. The pharmaceutical composition as in claim 20, wherein said compound is a complex of 2,4-dihydroxy-6-methylpyrimidine with N-methyl-D-glucamine and said concentration is from about 10 to about 500 micrograms per milliliter.

22. A method of blocking excess or inappropriate apoptosis in a mammal or a human in need of such treatment, said method comprising administering to said mammal or human an effective amount of 6-methyluracil based water soluble compound, or pharmaceutically acceptable solution thereof.

23. The method as in claim 22, wherein said compound is a complex of 2,4-dihydroxy-6-methylpyrimidine with N-methyl-D-glucamine.

24. The method as in claim 22, wherein said excessive or inappropriate apoptosis occurs in Alzheimer's disease.

25. The method as in claim 22, wherein said excessive or inappropriate apoptosis occurs in Parkinson's disease.

26. The method as in claim 22, wherein said excessive or inappropriate apoptosis occurs in viral infections.

27. The method as in claim 22, wherein said excessive or inappropriate apoptosis occurs during infarction or reperfusion injury.

28. The method as in claim 22, wherein said excessive or inappropriate apoptosis occurs during ischemia.

29. The method as in claim 22, wherein said excessive or inappropriate apoptosis results in excessive bone loss.

30. The method as in claim 22, wherein said excessive or inappropriate apoptosis results in hepatocellular degeneration.

31. A method of reducing proteolytic decay and general preservation of a food article including exposing said article to a 6-methyluracil based water-soluble compound during at least a portion of a storage period.

32. The method as in claim 31, wherein said compound is dissolved in a biocompatible water solution in a concentration from about 1 to about 1000 micrograms per milliliter.

33. The method as in claim 32, wherein said compound is a complex of 2,4-dihydroxy-6-methylpyrimidine with N-methyl-D-glucamine.

34. The method as in claim 31, wherein said food article is fish.

35. The method as in clam 34, wherein said compound is added to the salting solution in concentration of about 100 mg per kilogram of said fish.

36. The method as in claim 34, wherein said compound is a complex of 2,4-dihydroxy-6-methylpyrimidine with N-methyl-D-glucamine dissolved in water in concentration of about 1%.

37. The method as in claim 31, wherein said food article is caviar.

38. The method as in claim 31, wherein said food article is meat.

39. A method of protecting eukaryotic cells against radiation comprising a step of administering to said cells of a 6-methyluracil based water-soluble compound in a pharmaceutically acceptable form.

40. The method as in claim 39, wherein said compound is a complex of 2,4-dihydroxy-6-methylpyrimidine with N-methyl-D-glucamine.

41. A method of producing a complex of 2,4-dihydroxy-6-methylpyrimidine with N-methyl-D-glucamine consisting essentially of dissolving 2,4-dihydroxy-6-methylpyrimidine in an aqueous solution of equimolar amount of N-methyl-d-glucamine at a temperature from about 60 to about 70 degrees Celsius, followed by isolating of the desired product from the solution.

42. The method as in claim 41, wherein the step of isolating the desired product includes a step of concentrating the solution and co-evaporating thereof with 2-propanol.

43. The method as in claim 41, wherein the step of isolating includes crystallization with a solvent selected from a group consisting of water, propanol, ethanol, and isopropanol.
